(19) United States
(12) Patent Application Publication
(10) Pub. No.: US 2025/0263441 A1
(43) Pub. Date: Aug. 21, 2025

(54) PEPTIDE EXHIBITING ANTIBACTERIAL AND ANTI-INFLAMMATORY ACTIVITIES AND METHOD FOR TREATING MEDICAL CONDITION WITH THE SAME

(71) Applicant: Metanovas Biotech Inc., San Francisco, CA (US)

(72) Inventors: Qichang DONG, San Francisco, CA (US); Heng LUO, San Francisco, CA (US); Shaohua WANG, San Francisco, CA (US); Meijie WANG, San Francisco, CA (US); Lun YU, San Francisco, CA (US)

(21) Appl. No.: 19/059,206

(22) Filed: Feb. 20, 2025

Related U.S. Application Data
(63) Continuation-in-part of application No. 63/555,503, filed on Feb. 20, 2024.

(51) Int. Cl. C07K 9/00; A61K 38/00

(52) U.S. Cl. CPC C07K 9/00; A61K 38/00

(57) ABSTRACT

A peptide exhibiting both antibacterial and anti-inflammatory activities or a derivative thereof, including one of the following sequences: an amino acid sequence of SEQ ID NO: 1 or a variant thereof, and an amino acid sequence of SEQ ID NO: 2 or a variant thereof. The peptides synthesized in this disclosure exhibit antibacterial and anti-inflammatory effects, inhibiting one or more of the following: Escherichia coli, Staphylococcus aureus, Propionibacterium acnes, Candida albicans, as well as the expression of IL-6, IL-1B, CCL-2, IL-1, and NO.

### CROSS-REFERENCE TO RELATED APPLICATIONS

This application is a continuation-in-part of U.S. Ser. No. 63/555,503 filed on Feb. 20, 2024, now pending. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### INCORPORATION-BY-REFERENCE OF SEQUENCE LISTING

This application comprises a sequence listing, which has been submitted electronically in XML file and is incorporated herein by reference in its entirety. The XML file, created on Feb. 12, 2025, is named SZWK-00101-UUS.xml, and is 6,110 bytes in size.

### BACKGROUND

The present disclosure relates to the field of biomedical peptides, and more specifically, to a peptide exhibiting antibacterial and anti-inflammatory activities, and a method for treating medical condition with the same.

Antimicrobial peptides function as a natural host defense mechanism against exogenous pathogenic microorganisms. They disrupt bacterial plasma membrane structure, resulting in cell death. Inflammation is a defensive response of body tissues to injurious stimuli, including infection and tissue damage. Chronic inflammation is a key pathological basis for numerous diseases, such as arthritis, inflammatory bowel disease, and skin diseases.

Conventional peptides exhibiting both antibacterial and anti-inflammatory activities are biocompatible and can inhibit the growth of periodontitis-causing bacteria such as Porphyromonas gingivalis and Aggregatibacter actinomycetemcomitans, and inhibit bacterial biofilm formation. They are highly biocompatible and safe. However, these peptides are ineffective against bacteria such as Propionibacterium acnes, Staphylococcus aureus, Escherichia coli, and Candida albicans.

### SUMMARY

To solve the aforesaid problems, the disclosure provides a peptide or a derivative thereof, comprising one of the following sequences:
an amino acid sequence of SEQ ID NO: 1, or a variant thereof; and
an amino acid sequence of SEQ ID NO: 2, or a variant thereof.

In a class of this embodiment, the variant of SEQ ID NO: 1 or SEQ ID NO: 2 has ≥88% sequence identity with corresponding amino acid sequence.

In a class of this embodiment, the variant of SEQ ID NO: 1 has an amino acid sequence of SEQ ID NO: 3 or SEQ ID NO: 4.

In a class of this embodiment, variation(s) in SEQ ID NO: 1 consists of one or more conservative amino acid substitutions among amino acids within each of the following groups: (a) glycine, alanine, valine, leucine, and isoleucine; (b) phenylalanine, tyrosine, and tryptophan; (c) serine and threonine; (d) aspartate and glutamate; (e) glutamine and asparagine; and (f) lysine, arginine and histidine.

In a class of this embodiment, each of the amino acid sequences of SEQ ID NO: 1 and SEQ ID NO: 2 comprises an N-terminal; a hydrogen atom of the N-terminal is substituted by CH₃CH(OH)CO— or R₁—CO—, and R₁ is selected from a group consisting of H, hydroxyl, amino, an alkyl group, and an alkenyl group.

In a class of this embodiment, each of the amino acid sequences of SEQ ID NO: 1 and SEQ ID NO: 2 comprises a C-terminal; a hydrogen atom of the C-terminal is substituted by —NR₂R₃ or —OR₂, and R₂ and R₃ are independently selected from a group consisting of H, hydroxyl, amino, an alkyl group, and an alkenyl group.

In a class of this embodiment, the alkyl group is selected from a group consisting of methyl, ethyl, isopropyl, isobutyl, tert-butyl, pentyl, hexyl, heptyl, octyl, decyl, 2-ethylhexyl, 2-methylbutyl, and 5-methylhexyl.

In a class of this embodiment, the alkenyl group is selected from a group consisting of vinyl, linoleyl, and oleyl.

In another aspect, the disclosure provides a pharmaceutical composition, comprising: a medication container and a therapeutically effective amount of the peptide or a derivative thereof.

In a class of this embodiment, the peptide or a derivative thereof is in solid powder form in an amount of 200-2,500 mg per dose.

In a class of this embodiment, the pharmaceutical composition further comprises an aqueous solution contained in the medication container; the peptide or a derivative thereof is dissolved in the aqueous solution at a concentration of 3-30 mg/mL.

The disclosure further provides a method for treating a medical condition in a patient, comprising administering to the patient a therapeutically effective amount of the peptide or a derivative thereof.

In a class of this embodiment, an amount of the peptide or a derivative thereof is administered as a single dose is 200-2,500 mg per dose or 2-60 mg/kg of body weight.

In a class of this embodiment, the medical condition is acne and the peptide or a derivative thereof is administered topically to a treatment area on the patient's skin.

Further provided is a method for preparing a cosmetically or pharmaceutically acceptable salt comprising applying the peptide or a derivative thereof.

In a class of this embodiment, the salt is formed by the peptide or a derivative thereof of claim 1 with an organic base, and the organic base is ethylamine, diethylamine, arginine, lysine, histidine or piperazine.

In a class of this embodiment, the salt is formed by the peptide or a derivative thereof of claim 1 with an inorganic acid or an organic acid; the organic acid is acetic acid, citric acid, malonic acid, maleic acid, tartaric acid, fumaric acid, benzoic acid, succinic acid, oxalic acid, or gluconic acid; and the inorganic acid is hydrochloric acid, sulphuric acid, boric acid or carbonic acid.

The peptides synthesized in the disclosure have antibacterial and anti-inflammatory effects, inhibiting one or more of Escherichia coli, Staphylococcus aureus, Propionibacterium acnes, Candida albicans, as well as inhibiting the expression of IL-6, L-1b, CCL-2, IL-1 factor and nitric oxide (NO). The minimum inhibitory concentration of the peptides is less than the hemolytic concentration thereof and less than the maximum cytotoxicity-free concentration thereof, which makes it commercially available.

The synthesized peptides with antibacterial and anti-inflammatory effects of the disclosure have a minimum inhibitory concentration range of 50 g/mL against Escherichia coli, 12.5-50 g/mL against Staphylococcus aureus, 4-8 μg/mL against Propionibacterium acnes, and 25 g/mL against Candida albicans.

### BRIEF DESCRIPTION OF THE DRAWINGS

FIGS. 1A-1D show AMP5 reduces the release rates of the substance P, IL-1β, and IL-6 induced by TNF-α stimulation of SH-SY5Y cells, and does not affect the cell viability of SH-SY5Y cells according to one embodiment of the disclosure;

FIGS. 2A-2B show AMP5 inhibits the release rate of β-hexosaminidase and does not affect the cell viability of P815 mast cells;

FIG. 3 shows AMP33 does not inhibit the growth of Lactobacillus acidophilus;

FIGS. 4A-4B show after 16 h′ culture, AMP33 inhibited Streptococcus mutans at concentrations of 5 and 10 μg/mL, with an inhibition rate of about 100%;

FIGS. 5A-5B show after 48 h′ culture, 5 and 10 μg/mL of AMP33 exhibits good inhibitory activity against H. pylori;

FIG. 6 shows after 48 hours' culture, AMP33 with a concentration of 40 μg/mL does not affect the activity of Lactobacillus salivarius;

FIG. 7 shows AMP33 does not exhibit cytotoxicity at high concentrations; and FIG. 8 shows the peptide is not inactivated after high temperature treatment.

### DETAILED DESCRIPTION

To further illustrate the disclosure, embodiments detailing a peptide exhibiting both antibacterial and anti-inflammatory activities, and applications thereof are described below. It should be noted that the following embodiments are intended to describe and not to limit the disclosure.

In one aspect, the disclosure provides a therapeutic peptide (isolated). As used herein, “peptide” refers to a polypeptide consisting of 18 to 25 amino acid residues in length. The peptide may be produced by any suitable method, such as chemical synthesis. Any hydrogen atom in a peptide of the disclosure may be substituted with deuterium or tritium. The peptide could exhibit a net positive charge (i.e., cationic) in aqueous solution at pH 7.0.

### Defined Sequences:

SEQ ID NO: 1: GIFKKITGKLFKWIK (designated as AMP5).

SEQ ID NO: 2: LSKWLKKLGKLLAG (designated as AMP33).

SEQ ID NO: 3: GIFKKITGKCFKWIK, a variant of SEQ ID NO: 1 (designated as AMP5-L10C).

SEQ ID NO: 4: GIFKKIAGKLFKWIK, a variant of SEQ ID NO: 1 (designated as AMP5-T7A).

In some embodiments, the peptide comprises SEQ ID NO: 1 or SEQ ID NO: 2, or a variant thereof having ≥88% sequence identity with SEQ ID NO: 1 or 2, and optionally≥93% sequence identity therewith. For peptides comprising variants of SEQ ID NO: 1 or 2, there may be one or more conservative amino acid substitutions (in comparison to SEQ ID NO: 1). Such conservative amino acid substitutions may involve substitutions among amino acids within each of the following groups: (1) glycine, alanine, valine, leucine, and isoleucine; (2) phenylalanine, tyrosine, and tryptophan; (3) serine and threonine; (4) aspartate and glutamate; (5) glutamine and asparagine; and (6) lysine, arginine, and histidine. For example, the isoleucine at position 2 may be substituted with a leucine to create a variant of SEQ ID NO: 1 or 2.

The peptide of the disclosure may exhibit antimicrobial activity against one or more species of microbes (e.g., bacteria, fungi, parasites, etc.). The peptide may be effective against one or more Gram-positive bacteria, one or more Gram-negative bacteria, or both. Additionally, the peptide may be effective against both bacteria and fungi. Examples of bacteria against which the peptide may be effective include Candida albicans, Propionibacterium acnes, Escherichia coli, and Staphylococcus aureus.

The peptide of the disclosure may also possess anti-inflammatory activity. The peptide may reduce blood biomarkers of inflammation, such as C-reactive protein (CRP), lipid peroxidation products (e.g., prostaglandins), fibrinogen, and pro-inflammatory cytokines (e.g., IL-1, IL-6, TNF-α, etc.). Anti-inflammatory action may be indicated by blood biomarkers, such as a decrease in nitric oxide levels and an increase in anti-inflammatory cytokines (e.g., IL-10, IL-4, TGF-β, IL-1 receptor antagonist, etc.).

For therapeutic purposes, the peptide of the disclosure may be provided in any suitable form for delivery via any appropriate route of administration, such as injection, oral, topical, etc. In some embodiments, the peptide is provided in an aqueous solution that can be delivered by injection (e.g., intravenous or intramuscular). The concentration of the peptide in the aqueous solution may range from 3 to 30 mg/mL. The aqueous solution (containing the peptide) may be provided in a medication container as a single-use product. This medication container may be any suitable type of container for holding medications, such as a vial, ampoule, bottle, pre-filled syringe, bag, etc. The volume of the aqueous solution (containing the peptide) in the medication container may be≤30 mL, and optionally ≥1.0 mL.

In some embodiments, the peptide is provided in solid powder form (e.g., as a lyophilized powder) within a medication container as a single-use product. For use, the peptide (in powder form) is reconstituted into an aqueous solution and delivered via injection into the patient. In this context, the amount of peptide in the medication container may range from 200 to 1,200 mg.

In some embodiments, the peptide is provided in a topical composition. Examples include ointments, gels, creams, lotions, foams, pastes (e.g., toothpaste), skin patches, sprays, and aqueous solutions (e.g., mouthwash). In this context, the concentration of the peptide in the topical composition may range from 0.5 to 25 ppm (parts per million), and optionally from 2.0 to 10 ppm.

For convenient use, the topical composition may be provided in a topical medication dispenser (such as a tube or bottle). The medication dispenser may contain 20 to 250 grams of the topical composition, expressed in weight. For example, the product could be a dispensing tube containing 60 grams of the topical composition. In the context of a topical aqueous solution, the medication dispenser may contain 15 to 1,500 mL of the topical composition. For instance, the product could be a mouthwash solution with 1.0 liter contained in a bottle.

In another aspect, the disclosure relates to a method for treating a medical condition in a patient. Any relevant medical condition may be treated. For example, the medical condition could be acne. In this context, the peptide may be effective in treating (by topical application) both the bacterial infection (e.g., Propionibacterium acnes, Candida albicans, etc.) and the resulting inflammation. Other medical conditions that may be treated include additional pathogenic bacterial or fungal infections.

The method comprises administering a therapeutically effective amount of a peptide of the disclosure to the patient. The peptide may be administered to the patient by injection (e.g., intravenous or intramuscular). In some embodiments, the amount of peptide administered to the patient (as a single dose) ranges from 2 to 60 mg/kg of body weight. In other embodiments, the amount of peptide administered to the patient (as a single dose) ranges from 200 to 2,500 mg.

The peptide may also be administered topically, such as on the skin, mucosa, or in a body cavity (e.g., oral, nasal, anal, vaginal, etc.). In this context, the method may comprise applying 2.0 to 50 grams of the topical composition to the treatment area.

### Example 1

This example provides a peptide exhibiting both antibacterial and anti-inflammatory activities, and applications thereof, comprising polypeptide 1. Polypeptide 1 has the amino acid sequence shown in SEQ ID NO: 1: GIFKKITGKLFKWIK. Polypeptide 1 is synthesized as follows:

(1) The polypeptide is synthesized from the C-terminal to the N-terminal: 10 g of 2-chlorotrityl chloride resin is placed in a reaction tube, 15 mL/g of dichloromethane (DCM) is added, and the mixture is shaken for 30 minutes.

(2) Connecting the first amino acid: the solvent DCM is filtered off using sand core suction, Fmoc-Lys-OH in 3-fold molar excess relative to theoretical demand is added, and dimethylformamide (DMF) is further added for dissolution. Then, N,N-diisopropylethylamine (DIEA) in 6-fold molar excess relative to theoretical demand is added, and the mixture is shaken for 90 minutes. The resulting product is washed with methanol.

(3) Deprotection: spin evaporation is used to remove DMF, followed by the addition of 15 mL/g of 20% (v/v) piperidine-DMF solution for washing for 5 minutes. The piperidine-DMF solvent is then removed, and 15 mL/g of 20% (v/v) piperidine-DMF solution is added for washing for an additional 15 minutes.

(4) Detection: Remove the piperidine solution, take 15 grains of resin, wash with ethanol three times, add a detection reagent, and heat at 105-110° C. for 5 minutes to perform the detection. The appearance of a dark blue color of the solution indicates a positive reaction.

(5) Rinse resin: Rinse the resin twice with DMF (10 mL/g), DCM (10 mL/g), and then DMF (10 mL/g) in sequence.

(6) Condensation: Add the next amino acid derivative (from right to left of the peptide sequence) dissolved in three times the molar amount of DMF, along with HBTU (benzotriazole-N,N,N′,N′-tetramethyluronium hexafluorophosphate) also dissolved in three times the molar amount of DMF into the reaction tube. Immediately add DIEA in 10-fold molar excess relative to theoretical demand, and carry out the reaction for 30 minutes.

(7) Detection: Take 15 grains of resin, wash with ethanol three times, and add a detection reagent. Heat at 105-110° C. for 5 minutes to perform the detection. No color appearance indicates a negative reaction.

(8) Rinse the resin: Rinse the resin twice with DMF (10 mL/g), DCM (10 mL/g), and finally DMF (10 mL/g) in sequence.

(9) Repeat operations (3) to (8): Sequentially connect the amino acids in the order shown in SEQ ID NO: 1 from right to left.

(10) Evacuate and wash the resin: Wash the resin with DMF (10 mL/g) twice, methanol (10 mL/g) twice, DMF (10 mL/g) twice, DCM (10 mL/g) twice, and then evacuate for 10 minutes.

(11) Cutting peptides from the resin: The cutting solution composition was as follows: 95% (v/v) trifluoroacetic acid (TFA), 1% (v/v) water, 2% (v/v) mercaptoethanol (EDT), and 2% (v/v) triisopropylsilane (TIS). The cutting time was 120 minutes, resulting in the formation of a lysate.

(12) Blow-drying and washing: The lysate was blown dry with nitrogen and washed six times with ether, then allowed to volatilize at room temperature.

(13) Analyzing, purifying, and lyophilization: The crude peptide was purified by high-performance liquid chromatography. The peptide solution was collected into a lyophilizer for concentration, resulting in a white powder, which is a small molecule peptide represented by SEQ ID NO: 1.

The detection reagent used was ninhydrin.

Likewise, the peptides AMP5-L10C and AMP5-T7A were synthesized.

The AMP5, AMP5-L10C, and AMP5-T7A peptides were subjected to surface plasmon resonance (SPR) experiments to determine their binding affinity to Toll-Like Receptor 2 (TLR2). All three peptides exhibited high binding affinity to TLR2: for AMP5, KD=0.5432 μM; for AMP5-L10C, KD=0.8342 μM; and for AMP5-T7A, KD=0.3731 μM.

### Example 2

Example 2 is essentially the same as Example 1, except that the amino acid sequence of the polypeptide is shown in SEQ ID NO: 2: LSKWLKKLGKLLAG.

### Example 3

### Determination of Antimicrobial Effect of the Peptides

1. Inhibitory Effect of the Peptides on Candida albicans

### (i) Preparation of the Peptides

Primary screening of liquid culture: the peptides in Examples 1 to 2 were dissolved in pure water to a concentration of 100 μg/mL. Using a micropipette, 10 μL of the peptides were added to a new 96-well plate in order from left to right, starting from the second compartment. Positive and negative controls were established for use. The 96-well plates were arranged vertically, and the peptide concentrations of the control groups were the same as those of the antimicrobial peptide. The control groups consisted of: pure water and bacteria, antimicrobial peptide FK13 and bacteria, and antimicrobial peptide HPA3NT3 and bacteria. An aseptic medium was used. The amino acid sequence of FK13 is FPLTWLKWWKWKKK (SEQ ID NO: 5), and the amino acid sequence of HPA3NT3 is FKRLKKLFKKKKIWNWK (SEQ ID NO: 6).

The 96-well plates containing peptides of different concentrations were diluted to concentrations of 100 μg/mL, 50 μg/mL, 25 μg/mL, 12.5 μg/mL, 8 μg/mL, 4 μg/mL, 2 μg/mL, 1 μg/mL, 0.5 μg/mL, and 0.25 μg/mL for future use.

If a 96-deep-well plate was used to culture bacteria, an additional 100 μL of sterile medium was added to all wells.

If a 96-deep-well plate was used to culture bacteria, an additional 100 μL of sterile medium was added to all wells.

### (ii) Bacterial Recovery and Bacterial Suspension Preparation

Candida albicans was inoculated in LB medium with normal oxygen concentration (21%) for 48 hours. E. coli clones were picked and incubated in LB medium at 37° C. with normal oxygen concentration (21%) for 48 hours. The bacterial concentration was measured and diluted to 107 CFU/mL with culture medium for use.

(iii) Detection of the Minimum Inhibitory Concentration of Peptides Using Liquid Culture

The prepared fresh bacterial suspension was diluted to 105 CFU/mL and added to the 96-well plate with 90 μL of the bacterial suspension per well, and incubated at 37° C. with normal oxygen concentration for 24 to 48 hours. The absorbance at 630 nm was measured with a microplate reader, and the degree of turbidity was observed by the naked eye. The lowest concentration without turbidity was determined as the minimum inhibitory concentration of the peptide under the gradient concentrations of 100 μg/mL, 50 μg/mL, 25 μg/mL, 12.5 μg/mL, 8 μg/mL, 4 μg/mL, 2 μg/mL, 1 μg/mL, 0.5 μg/mL, and 0.25 μg/mL, respectively.

(iv) Repetition of Operations: The Operations in (iii) were Repeated Twice, and the Decision to Perform a Third Repetition was Based on the Reproducibility of the Experiment.

The LB medium was purchased from Zhongke Ruitai (Beijing) Biotechnology Co

The results indicate that the peptides inhibited Candida albicans, as shown in Table 1. In the same operational system, the inhibitory effect of the peptide on Candida albicans was found to be more effective at a concentration of 25 μg/mL.

TABLE 1

| Minimum | Minimum | Minimum | Minimum |
|---|---|---|---|
| inhibitory | inhibitory | inhibitory | inhibitory |
| concentration | concentration | concentration | concentration |
| on | on | on | on |
| Escherichia | Staphylococcus | Propionibacterium | Candida albicans |
| coli (μg/mL) | aureus (μg/mL) | acnes (μg/mL) | (μg/mL) |
| 50 | 50 | 8 | 25 |
| 50 | 12.5 | 4 | 25 |
| 0.25 | 0.25 | 0.25 | / |
| 50 | 4 | 4 | 25 |
| 25 | 4 | 2 | 50 |

2. Determination of the Inhibitory Effect of Peptides on Escherichia coli, Staphylococcus aureus, and Propionibacterium acnes

### (i) Preparation of the Peptides

Primary screening of liquid culture: the peptides in Examples 1 to 2 were dissolved in pure water to a concentration of 100 μg/mL. Using a micropipette, 10 μL of the peptides were added to a new 96-well plate in order from left to right, starting from the second compartment. Positive and negative controls were established for use. The 96-well plates were arranged vertically, and the peptide concentrations in the control groups were the same as those of the antimicrobial peptides. The control groups included: pure water and bacteria, antimicrobial peptide FK13 and bacteria, antimicrobial peptide HPA3NT3 and bacteria, and ciprofloxacin and bacteria. An aseptic medium was used.

96-well plates containing 10 μL of peptides at different concentrations were diluted to final concentrations of 100 μg/mL, 50 μg/mL, 25 μg/mL, 12.5 μg/mL, 8 μg/mL, 4 μg/mL, 2 μg/mL, 1 μg/mL, 0.5 μg/mL, and 0.25 μg/mL for future use.

If a 96-deep-well plate was used to culture bacteria, an additional 100 μL of sterile medium was added to all wells.

### (ii) Bacterial Recovery and Bacterial Suspension Preparation

E. coli was inoculated in LB medium with normal oxygen concentration (21%) for 48 hours. E. coli clones were picked and incubated in LB medium at 37° C. with normal oxygen concentration (21%) for an additional 48 hours. The bacterial concentration was measured and diluted to 107 CFU/mL with culture medium for use.

Inoculation and Culture of Staphylococcus aureus

Staphylococcus aureus was inoculated in LB medium with a normal oxygen concentration (21%) for 48 hours. Staphylococcus aureus clones were picked and incubated in LB medium at 37° C. with a normal oxygen concentration (21%) for an additional 48 hours. The bacterial concentration was measured and diluted to 107 CFU/mL with culture medium for use.

Inoculation and Culture of Propionibacterium acnes

Propionibacterium acnes was inoculated on an anaerobic blood agar plate, placed in an anaerobic bag, and incubated anaerobically at 37° C. for 48 hours. Propionibacterium acnes clones were then picked and incubated anaerobically in liquid anaerobic broth medium at 37° C. for an additional 48 hours. The bacterial concentration was measured and diluted to 107 CFU/mL with culture medium.

(iii) Detection of the Minimum Inhibitory Concentration of Peptides Using Liquid Culture

The prepared fresh bacterial suspension was diluted to 105 CFU/mL and added to the 96-well plate with 90 μL of the bacterial suspension per well. The plate was incubated at 37° C. with a normal oxygen concentration for 24 to 48 hours (Propionibacterium acnes was cultured anaerobically at 37° C. for 24 to 48 hours in an anaerobic bag). The absorbance at 630 nm was measured using a microplate reader, and the degree of turbidity was observed by the naked eye. The lowest concentration without turbidity was determined as the minimum inhibitory concentration of the peptide under the gradient concentrations of 100 μg/mL, 50 μg/mL, 25 μg/mL, 12.5 μg/mL, 8 μg/mL, 4 μg/mL, 2 μg/mL, 1 μg/mL, 0.5 μg/mL, and 0.25 μg/mL, respectively.

(iv) Repetition of Operations: The Operations in (iii) were Repeated Twice, and the Decision to Perform a Third Repetition was Based on the Reproducibility of the Experiment.

The LB medium was purchased from Zhongke Ruitai (Beijing) Biotechnology Co.

The anaerobic blood agar plate was a CDC anaerobic blood agar plate.

The CDC anaerobic blood agar plate was purchased from Henan Meikai Biotechnology Co.

The inhibitory effects of the polypeptides on Escherichia coli, Staphylococcus aureus, and Propionibacterium acnes are shown in Table 1. Under the same experimental conditions, the inhibitory effects of the peptides from Examples 1 and 2 on E. coli were both 50 μg/mL, on S. aureus were 50 μg/mL and 12.5 μg/mL, respectively, and on Propionibacterium acnes were 8 μg/mL and 4 μg/mL, respectively.

In Table 1, “/” indicates that the corresponding assays were not performed for the respective strains.

### 3. Measurement of Hemolytic Concentration

4 mL of 2% rabbit blood erythrocytes were washed twice with 4 mL PBS and centrifuged at 3000 rpm for 5 minutes. The supernatant was discarded, and the rabbit blood erythrocytes were resuspended in 4 mL PBS. 160 μg of the peptides from Examples 1 to 2, FK13, and HPA3NT3 were independently dissolved in 1 mL PBS buffer to prepare the peptide mother solutions with a concentration of 160 μg/mL. Subsequently, the peptide mother solutions were diluted with PBS buffer to concentrations of 80 μg/mL, 64 μg/mL, 32 μg/mL, 16 μg/mL, and 8 μg/mL. 100 μL of the 2% rabbit blood erythrocytes and 100 μL of different concentrations of peptides (80 μg/mL, 64 μg/mL, 32 μg/mL, 16 μg/mL, 8 μg/mL) were added to each well of a 96-well plate. In the negative control, 100 μL of PBS buffer was added, while in the positive control, 100 μL of 0.1% (w/v) Triton X-100 was added. Three replicate wells were set up for each group and incubated at 37° C. for 1 hour. The 96-well plate was then removed, centrifuged at 3000 rpm for 5 minutes, and 100 μL of supernatant was aspirated and transferred to a new 96-well plate. The absorbance was measured at 540 nm using an enzyme meter. Hemolysis rate (%)=((Absorbanceₛₐₘₚₗₑ−Absorbance_{negative})/(Absorbanceₚₒₛᵢₜᵢᵥₑ−Absorbance_{negative}))*100%.

The test results of peptide hemolysis concentration are shown in Table 2.

TABLE 2

| | Example 1 | Example 2 | FK13 | HPA3NT3 |
|---|---|---|---|---|
| 80 μg/mL | 14.6 | 10.1 | 27.0 | 9.7 |
| | −0.9 | 10.1 | 21.5 | 8.5 |
| | 2.8 | 11.1 | 27.4 | 13.7 |
| 64 μg/mL | 3.7 | 0.7 | 5.3 | 21.4 |
| | −1.0 | −0.8 | 2.5 | −0.8 |
| | 3.8 | −2.0 | 2.7 | 0.6 |
| 32 μg/mL | 62 | 0.6 | 2.5 | 2.7 |
| | 2.8 | 4.6 | 3.1 | 1.4 |
| | −1.9 | 3.3 | 5.0 | 7.8 |
| 16 μg/mL | 5.9 | 2.6 | 3.4 | 23 |
| | 1.8 | 1.7 | 2.5 | −0.2 |
| | 80.0 | −0.4 | 0.4 | 0.4 |
| 8 μg/mL | 14.6 | 3.6 | 3.9 | 2.4 |
| | −0.9 | 7.9 | 7.2 | 6.2 |
| | 2.8 | 23 | 5.9 | 4.7 |
| Minimum hemolytic | 3.7 | 64.0 | 64.0 | 64.0 |
| concentration (μg/mL) | | | | |

### 4. Cytotoxic Assay

Preparation of Cell Suspension: HaCat (human immortalized epidermal cells) cells were counted and inoculated into 96-well plates. 100 μL of cell suspension was added to each well, corresponding to a density of 5×10⁴ cells per well, with three replicates for each sample. The cells were cultured in a 37° C. incubator for 4 hours until they were attached to the plate. Peptides from the examples, as well as FK13 and HPA3NT3, were then added at concentrations of 80 μg/mL, 32 μg/mL, and 8 μg/mL. The cells were incubated at 37° C. for 60 hours. Afterward, 10 μL of CCK-8 solution was added, and the cells were cultured for an additional 2 hours. The absorbance of the peptides at 450 nm was measured using a dual-wavelength detector, with a detection wavelength of 450-490 nm and a reference wavelength of 600-650 nm. The maximum cytotoxicity-free concentration is shown in Table 3.

TABLE 3

| | Maximum cytotoxicity-free |
|---|---|
| Example | concentration (μg/mL) |
| Example 1 | 80 |
| Example 2 | 80 |
| FK13 | 80 |
| HPA3NT3 | 32 |

From Tables 1-3, it can be seen that the peptides from the examples can simultaneously inhibit Escherichia coli, Staphylococcus aureus, Propionibacterium acnes, and Candida albicans, demonstrating excellent antibacterial effects across various spectra.

### Example 4

### The Anti-Inflammatory Effect of AMP5

### 1. The Effect of AMP5 on the Protein Expression of Substance P, IL-1B, and IL-6 Induced by SH-SY5Y Cells

### Experimental Steps:

1) HaCaT cells were cultured in DMEM with 10% FBS and 1% P/S. SH-SY5Y cells were cultured in DMEM supplemented with 2 mM glutamine, 10% FBS, and 1% P/S.

2) The cultured SH-SY5Y cells were seeded at 20,000 cells/well and cultured for 24 hours. The supernatant was removed, and the cells were treated with 10 μM retinoic acid for 4 days to induce neuronal differentiation. Successful differentiation of SH-SY5Y cells was observed using a microscope.

3) After differentiation, the supernatant was discarded, and the cultured HaCaT cells (20,000 cells/well) were added to the differentiated SH-SY5Y cells for co-culturing in DMEM for 2 days.

4) Following successful co-culturing of the two cell types, the supernatant was discarded, and the cells were further cultured for 24 hours. The co-cultured cells were then used as experimental cells. Based on the grouping settings, the co-cultured cells were stimulated with 10 ng/mL TNF-α and treated with AMP5 at a concentration of 50 μg/mL. After 24 hours of stimulation, the supernatant was collected for testing.

5) An ELISA kit was employed to measure the levels of substance P, IL-1B, and IL-6 in the supernatant of different groups, with 2 replicates for each measurement.

6) 150 μL of culture medium and 10 μL of CCK-8 were added to each well of the lower layer cells to measure cell viability. After 2 hours, the optical density (OD) at 450 nm was measured. The calculation formula for cell viability is:

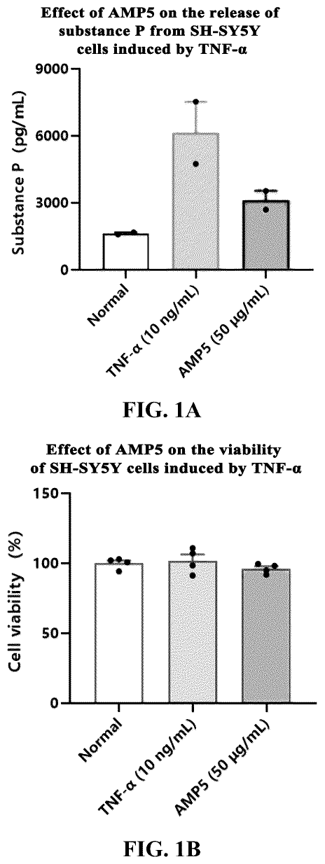

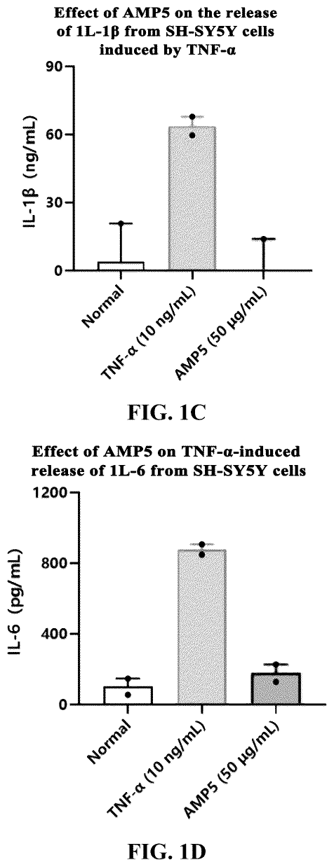

As shown in FIGS. 1A-1D, the results indicate that AMP5 reduced the release rates of substance P, IL-1B, and IL-6 induced by TNF-α stimulation of SH-SY5Y cells, without affecting cell viability. 2 hours later, OD₄₅₀ₙₘ was measured. The calculation formula for cell viability is: ((OD_{AMP5}−OD_{medium})/(OD_{Normal}−OD_{medium}))×100% (4 repeated wells).

### 2. The Effect of AMP5 on β-Hexosaminidase

### Experimental Steps:

1) Cell Culture of P815 Mast Cell Tumor in Mice: P815 cells were cultured in DMEM high-glucose medium (containing 10% fetal bovine serum, 2 mM glutamine, and 1% penicillin/streptomycin) at 37° C. in a 5% CO₂ incubator. The logarithmic growth phase P815 cells were collected for subsequent experiments.

2) The P815 cells were plated in a 96-well plate at a density of 20,000 cells per well. After allowing the cells to adhere for about 1 hour, an inhibitor (AMP5/sodium tryptophan SC) was added, followed by the addition of the inducer SP (0.1 μM) for 24 hours. The solution was then centrifuged, and the supernatant was collected for further tests.

3) The operation for determining the content of β-hexosaminidase was carried out according to the instructions of the reagent kit. The calculation of the β-hexosaminidase release rate is given by the formula: A450 (AMP5)/A450 (Normal)×100%.

4) 150 μL of culture medium and 10 μL of CCK-8 were added to each well of the lower layer cells to measure cell viability. After 2 hours, the OD at 450 nm was measured. The calculation formula for cell viability is: 2 hours later, the OD_{450 nm} was measured. The calculation formula for cell viability is: ((OD AMP5-OD_{medium})/(OD_{Normal}-OD_{medium}))×100% (4 repeated wells).

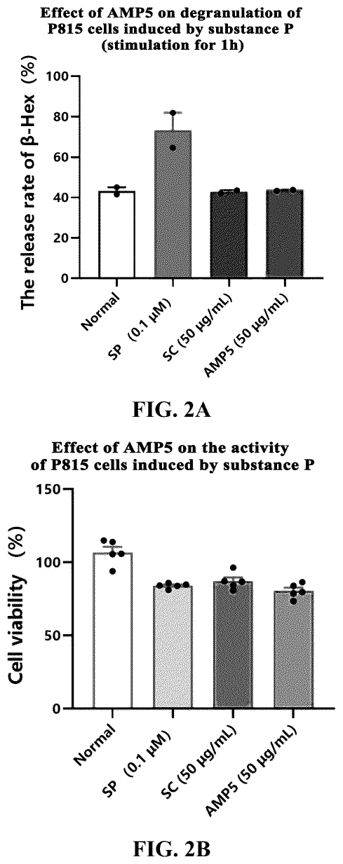

As shown in FIGS. 2A-2B, AMP5 inhibited the release rate of β-hexosaminidase without affecting cell viability.

### Example 5

### The Antibacterial Effect of AMP-33

1. The Inhibitory Effect of AMP-33 on Propionibacterium acnes (ATCC: 6919)

### Experimental Steps:

(1) Propionibacterium acnes was inoculated into Wilkins Chalgren liquid medium and cultured overnight under anaerobic conditions at 37° C. (Labiophy HL-B fully automated anaerobic workstation).

(2) 200 μL of the Wilkins Chalgren liquid culture medium and a predetermined concentration of the peptide AMP-33 were added to a 96-deep-well plate. Then, 10 μL of Propionibacterium acnes was inoculated (mix well before inoculation).

(3) After 48 hours of cultivation under anaerobic conditions at 37° C., the turbidity of the bacterial solution was observed. Then, 100 μL of the solution was taken, and the OD at 630 nm was measured using the enzyme-linked immunosorbent assay (ELISA) reading plate (after multiple blows and mixing).

$$Calculate the Percentage of Bacterial Viability = ( OD measurement - ODneg ) / ( ODpos - ODneg ) * 100 \qquad ( 4 )$$

OD Measurement: OD value of the test peptide.
ODneg: OD value with no bacterial inoculation, containing only LB medium.
ODpos: OD value with bacterial inoculation and without inhibitors.

TABLE 4

| | |
|---|---|
| Peptide concentration | Viability of Propionibacterium acnes |

| | | | | |
|---|---|---|---|---|
| 100 | μg/mL | −0.85 | −0.16 | −0.43 |
| 50 | μg/mL | −0.98 | 0.25 | −0.91 |
| 25 | μg/mL | −1.25 | 0.11 | 0.09 |
| 12.5 | μg/mL | −0.98 | 0.11 | −0.75 |
| 8 | μg/mL | −0.98 | 0.11 | 0.09 |
| 4 | μg/mL | −0.98 | −0.03 | 7.78 |
| 2 | μg/mL | 79.37 | 65.48 | 68.87 |
| 1 | μg/mL | 94.82 | 113.75 | 94.03 |

Table 4 shows the results of three repeated experiments, indicating that AMP-33 can inhibit Propionibacterium acnes by over 90% at low concentrations (4 ppm).

2. The Inhibitory Effect of AMP-33 on Escherichia coli (ATCC: 25922) and Staphylococcus aureus (ATCC: 25913)

(1) Staphylococcus aureus and Escherichia coli were inoculated into LB liquid medium and cultured overnight under aerobic conditions at 37° C. and 1800 rpm on a shaker.

(2) 200 μL of LB liquid culture medium and a predetermined concentration of peptide AMP-33 were added to a 96-deep-well plate. Then, 10 μL of the Staphylococcus aureus/Escherichia coli solution was inoculated (mixed well before inoculation).

(3) After incubating at 1800 rpm for 24 hours on a shaking table at 37° C., the turbidity of the bacterial solution was observed. A volume of 100 μL of the solution was taken, and the OD at 630 nm was measured using an enzyme-linked immunosorbent assay (ELISA) reader (after multiple blows and mixing).

$$Calculate the Percentage of Bacterial Viability = ( OD measurement - ODneg ) / ( ODpos - ODneg ) * 100. \qquad ( 4 )$$

OD Measurement: OD value of the test peptide.
ODneg: OD value with no bacterial inoculation, containing only LB medium.
ODpos: OD value with bacterial inoculation and without inhibitors.

TABLE 5

| Peptide | | Viability of | | | Viability of | | |
|---|---|---|---|---|---|---|---|
| concentration | | Staphylococcus aureus | | | Escherichia coli | | |
| 100 | μg/mL | −1.32 | −0.15 | 4.98 | −1.12 | 0.41 | −0.3 |
| 50 | μg/mL | −0.80 | −0.59 | 0.86 | 26.71 | 0.41 | 5.8 |
| 25 | μg/mL | −0.15 | −0.15 | 0.00 | 74.99 | 38.97 | 51.39 |
| 12.5 | μg/mL | 92.99 | 110.19 | 123.65 | 66.55 | 53.44 | 48.27 |
| 8 | μg/mL | 98.53 | 99.19 | 97.03 | 78.87 | 50.46 | 73.2 |
| 4 | μg/mL | 88.75 | 109.74 | 132.77 | 86.48 | 70.83 | 70.35 |
| 2 | μg/mL | 121.60 | 104.84 | 102.77 | 89.71 | 90.4 | 98.53 |
| 1 | μg/mL | 117.78 | 116.41 | 116.45 | 110.11 | 91.93 | 82.94 |

Table 5 shows the results of three repeated experiments, indicating that AMP-33 can inhibit over 99% of Escherichia coli at a concentration of 25 ppm and Staphylococcus aureus at a concentration of 50 ppm.

3. The Inhibitory Effect of AMP-33 on Candida albicans (ATCC: 14053)

(1) Candida albicans was inoculated into YM liquid medium and cultured overnight under aerobic conditions at 28° C. and 1800 rpm on a shaker.

(2) 200 μL of YM liquid culture medium and a predetermined concentration of peptide AMP-33 were added to a 96-deep-well plate. Then, 10 μL of the Candida albicans solution was inoculated (mixed well before inoculation).

(3) After incubating at 1800 rpm for 28 hours on a shaking table at 28° C., the turbidity of the bacterial solution was observed. A volume of 100 μL of the solution was taken, and the OD at 630 nm was measured using an enzyme-linked immunosorbent assay (ELISA) reader (after multiple blows and mixing).

$$Calculate the Percentage of Bacterial Viability : = ( OD measurement - ODneg ) / ( ODpos - ODneg ) * 100 \qquad ( 4 )$$

OD Measurement: OD value of the test peptide.
ODneg: OD value with no bacterial inoculation, containing only LB medium.
ODpos: OD value with bacterial inoculation and without inhibitors.

TABLE 6

| | |
|---|---|
| Peptide | |
| concentration | Viability of Candida albicans |

| | | | | |
|---|---|---|---|---|
| 50 | μg/mL | −2.09732 | −0.20973 | −0.71309 |
| 25 | μg/mL | −1.46812 | −0.96477 | −1.7198 |
| 12.5 | μg/mL | −1.34228 | −1.97148 | −1.84564 |
| 8 | μg/mL | 53.84082 | 70.73078 | 66.16615 |

Table 6 shows the results of three repeated experiments, indicating that AMP-33 can inhibit Candida albicans by over 99% at a concentration of 12.5 ppm.

4. Inhibition of AMP-33 on Lactobacillus acidophilus (ATCC: 4356)

### Experimental Steps:

(1) Lactobacillus acidophilus was inoculated into MRS liquid medium and cultured overnight under anaerobic conditions at 37° C. (Labiophy HL-B fully automated anaerobic workstation).

(2) 100 μL of MRS liquid culture medium and a predetermined concentration of peptide were added to a 96-deep-well plate. Then, 100 μL of Lactobacillus acidophilus was inoculated (mixed well before inoculation).

(3) After 48 hours of cultivation under anaerobic conditions at 37° C., the turbidity of the bacterial solution was observed. A volume of 100 μL of the solution was taken, and the OD at 630 nm was measured using an enzyme-linked immunosorbent assay (ELISA) reading plate (after multiple blows and mixing).

$$Calculate the Percentage of Bacterial Viability = ( OD measurement - ODneg ) / ( ODpos - ODneg ) * 100 \qquad ( 4 )$$

OD Measurement: OD value of the test peptide.
ODneg: OD value with no bacterial inoculation, containing only LB medium.
ODpos: OD value with bacterial inoculation and without inhibitors.

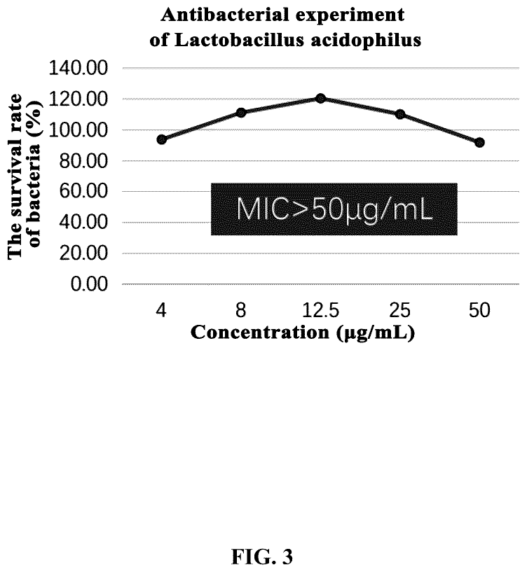

The results are shown in FIG. 3, indicating that AMP-33 does not inhibit the growth of Lactobacillus acidophilus.

5. The Inhibitory Effect of AMP-33 on Streptococcus mutans

### Experimental Steps:

(1) Streptococcus mutans was cultured to the logarithmic phase, then 5 mL of the bacterial solution was centrifuged at 5,000 g for 10 minutes. The organisms were resuspended in Brain-Heart Infusion (BHI) medium, and the concentration of the bacterial solution was adjusted to approximately 2×10⁵ CFU/mL using pre-configured McClure's turbidimetric tubes.

(2) Streptococcus mutans was cultured in a microtiter plate (96 wells), and 50 μL of different AMP33 peptides were added (at concentrations of 5 and 10 μg/mL). Then, 100 μL of diluted Streptococcus mutans bacterial fluid was added, followed by 50 μL of BHI medium, resulting in a final concentration of 1×10⁵ CFU/mL.

(3) A blank control was provided with equal amounts of medium solution and bacterial solution as that of the experimental group, with at least 3 replicates for each treatment group.

(4) The 96-well plate was placed in a bacterial incubator at 37° C. and incubated at 150 rpm for 16 hours. The OD at 600 nm represents the concentration of the bacterial solution, and the inhibition rate is calculated using the following formula: Inhibition rate (%)=(OD₆₀₀ control bacterial solution-OD₆₀₀ peptide-treated bacterial solution)/OD₆₀₀ control bacterial solution)×100%.

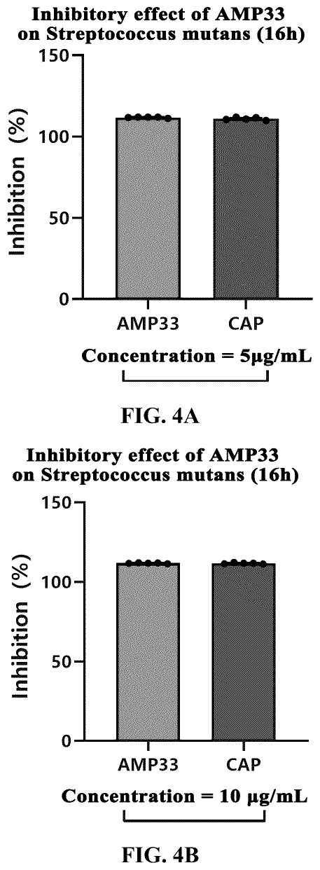

The results are shown in FIGS. 4A-4B, indicating that after 16 hours of culture, AMP33 inhibited Streptococcus mutans at concentrations of 5 and 10 μg/mL, with an inhibition rate of approximately 100%.

6. The Inhibitory Effect of AMP-33 on Helicobacter pylori
Experimental steps:

(1) Helicobacter pylori was cultured to the logarithmic phase. Then, 4.5 mL of the bacterial solution was centrifuged at 5,000 g for 6 minutes, and the organisms were resuspended in Wilkins-Chalgren medium. The concentration of the bacterial solution was adjusted to approximately 2× 105 CFU/mL using pre-configured McClure's turbidimetric tubes.

(2) Helicobacter pylori was cultured in a microtiter plate (96 wells), and 50 μL of different AMP33 peptides were added (at concentrations of 5 and 10 μg/mL). Next, 100 μL of diluted Helicobacter pylori bacterial fluid was added, followed by 50 μL of medium until the final concentration reached 1×10⁵ CFU/mL.

(3) A blank control was provided with equal amounts of medium solution and bacterial solution as in the experimental group, with at least 3 replicates for each treatment group.

(4) The 96-well plate was placed in a bacterial incubator at 37° C. and incubated at 150 rpm for 48 hours. The OD at 600 nm represents the concentration of the bacterial solution, and the inhibition rate is calculated using the following formula: Inhibition rate (%)=(OD₆₀₀ control bacterial solution-OD₆₀₀ peptide-treated bacterial solution)/OD₆₀₀ control bacterial solution)×100%.

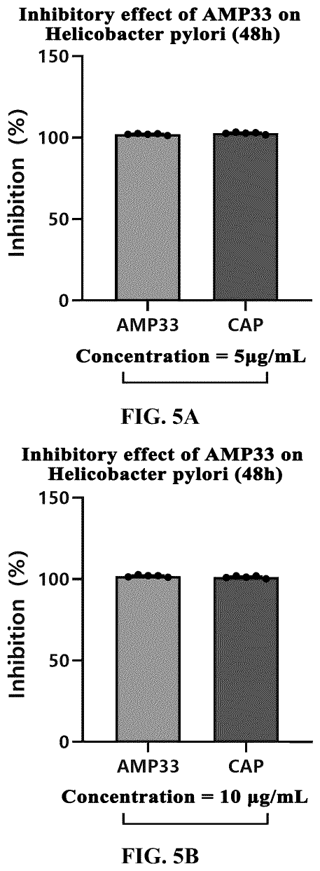

The results are shown in FIGS. 5A-5B, indicating that after 48 hours of culture, 5 and 10 μg/mL of AMP33 exhibited good inhibitory activity against H. pylori, approaching approximately 100%.

7. The Inhibitory Effect of AMP-33 on Lactobacillus salivarius

### Experimental Steps:

(1) Lactobacillus salivarius was cultured to the logarithmic phase. Then, 4.5 mL of the bacterial solution was centrifuged at 5,000 g for 6 minutes, and the organisms were resuspended in Wilkins-Chalgren medium. The concentration of the bacterial solution was adjusted to approximately 2× 105 CFU/mL using pre-configured McClure's turbidimetric tubes.

(2) Lactobacillus salivarius was cultured in a microtiter plate (96 wells), and 50 μL of different AMP33 peptides were added (at concentrations of 5 and 10 μg/mL). Next, 100 μL of diluted Lactobacillus salivarius bacterial fluid was added, followed by 50 μL of medium, until the final concentration reached 1×10⁵ CFU/mL.

(3) A blank control was provided with equal amounts of medium solution and bacterial solution compared to the experimental group, with at least 3 replicates for each treatment group.

(4) The 96-well plate was placed in a bacterial incubator at 37° C. and incubated at 150 rpm for 48 hours. The OD at 600 nm represents the concentration of the bacterial solution, and the inhibition rate is calculated according to the following formula:

$$Inhibition rate ( \% ) = ( {OD}_{600} control bacterial solution -$$

$${OD}_{600} peptide - treated bacterial solution ) /$$

$${OD}_{600} control bacterial solution ) \times 100 \% .$$

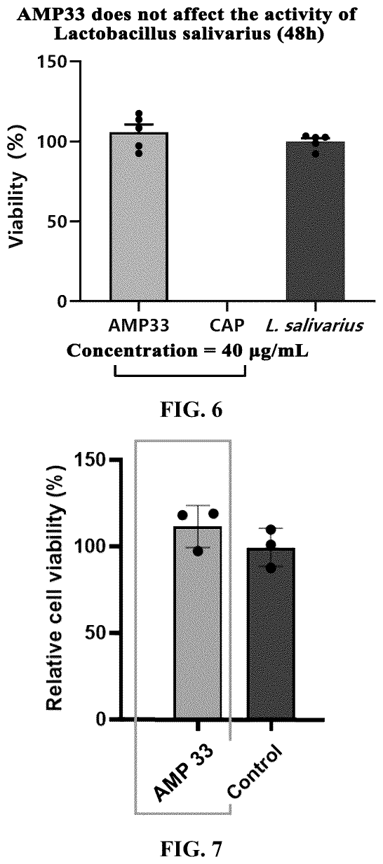

The results are shown in FIG. 6, indicating that after 48 hours of culture, AMP33 at a concentration of 40 μg/mL does not affect the activity of Lactobacillus salivarius.

### Example 6

### Cytotoxicity of AMP-33 Against RAW264.7

### Experimental Steps:

(1) Mouse macrophage RAW264.7 cells were cultured in DMEM supplemented with 10% fetal bovine serum (FBS) and 1% penicillin and streptomycin (P/S) at 37° C. in a 5% CO₂ incubator.

(2) 5×10⁴ mouse-derived RAW264.7 macrophages were placed in a 12-well cell culture plate and cultured for 24 hours. Then, 20 μL of PBS (negative control) and the test peptide were added to the wells, respectively, for incubation for 24 hours.

(3) CCK-8 reagent was added and incubated at 37° C. in a 5% CO₂ incubator for 2 hours. The OD at 450 nm was measured, and cell viability was calculated.

$$Viability = ( {OD}_{test} - {OD}_{neg} ) / ( {OD}_{pos} - {OD}_{neg} ) * 100$$

ODtest: OD value of the test peptide.
ODneg: OD value of medium without cells.
ODpos: OD value of the solvent (water).

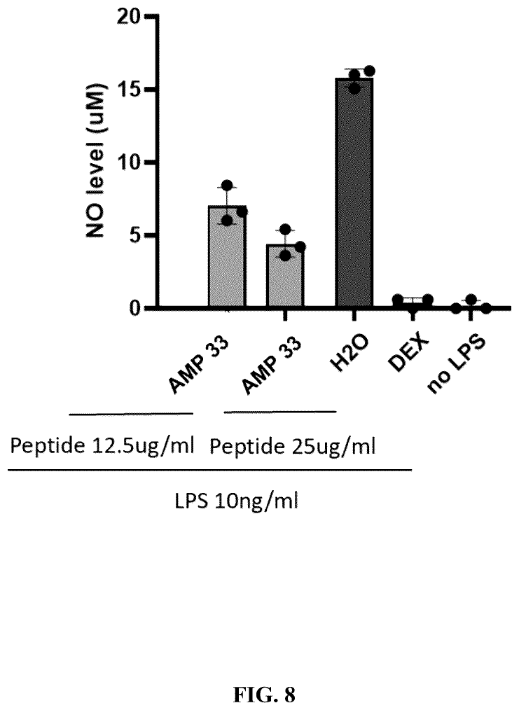

The results are shown in FIG. 7, indicating that AMP-33 does not exhibit cytotoxicity at high concentrations and is a safe raw material.

### Example 7

### Anti-Inflammatory Effect of AMP-33 on Mouse Macrophage RAW264.7 Cells

### Experimental Steps:

(1) Mouse macrophage RAW264.7 cells were cultured in DMEM supplemented with 10% fetal bovine serum (FBS) and 1% penicillin and streptomycin (P/S) at 37° C. in a 5% CO₂ incubator.

(2) 5×10⁴ mouse-derived RAW264.7 macrophages were placed in 12-well cell culture plates and cultured for 24 hours. Then, 20 μL of PBS (negative control), Dexamethasone (positive control), and the test peptide were added to the wells for incubation for 2 hours. Afterward, 10 ng/ml of lipopolysaccharide (LPS) was mixed with RAW264.7 cells and incubated for 24 hours.

(3) The supernatant was collected, and NO and IL-6/IL-1ß expression levels were measured using a commercial kit.

(4) The cells were collected, and total RNA was isolated and extracted, reverse transcribed, and subjected to RT-qPCR to detect inflammation markers, including TNF-α, IL-1β, IL-6/10, CCL2, IFN-γ, iNOS, and GAPDH.

1. NO Concentration After Treatment of RAW264.7 Cells with Peptide AMP-33

Table 7 shows the results of three replicate experiments:

TABLE 7

NO concentration (μM)

| | | | | |
|---|---|---|---|---|
| 100 | μg/mL | 1.60 | 1.13 | 1.54 |
| 50 | μg/mL | 1.27 | 1.33 | 1.60 |
| 25 | μg/mL | 2.82 | 2.89 | 3.83 |
| 12.5 | μg/mL | 5.79 | 6.53 | 6.67 |
| 8 | μg/mL | 10.26 | 10.75 | 11.10 |
| 4 | μg/mL | 12.35 | 12.14 | 11.10 |
| 2 | μg/mL | 12.70 | 13.54 | 13.37 |

| | | | |
|---|---|---|---|
| PBS | 17.45 | 17.87 | 17.47 |
| Dexamethasone | 6.80 | 6.06 | 3.43 |

2. After treating RAW264.7 cells with peptide AMP-33, an ELISA kit was used to detect the concentration of IL-6 and IL-1β proteins.

Table 8 shows the results of two repeated experiments:

TABLE 8

| | | |
|---|---|---|
| pg/mL | IL-1β | IL-6 |

| | | | | |
|---|---|---|---|---|
| 12.5 μg/mL AMP-33 | 56.2 | 32.6 | 46.4 | 62.3 |
| PBS | 200.1 | 199.8 | 119.4 | 95.3 |
| Dexamethasone | 75.1 | 44 | 14.2 | 23.9 |

3. After treating RAW264.7 cells with AMP-33, RT-qPCR was used to detect the levels of IL-6 and IL-1ß mRNA.

Table 9 shows the results of three repeated experiments.

TABLE 9

| | AMP-33 | | | PBS | | | Dexamethasone | | |
|---|---|---|---|---|---|---|---|---|---|
| IL-6 | 0.96 | 0.49 | 0.95 | 0.98 | 0.89 | 1.13 | 0.17 | 0.21 | 0.08 |
| IL-1β | 0.40 | 0.45 | 0.77 | 0.94 | 1.09 | 0.95 | 0.08 | 0.09 | 0.20 |
| CCL-2 | 0.51 | 0.52 | 0.58 | 1.19 | 1.05 | 0.76 | 0.37 | 0.38 | 1.65 |
| COX-2 | 0.71 | 0.58 | 1.16 | 0.84 | 0.93 | 1.33 | 0.37 | 0.34 | 1.53 |
| IL-10 | 1.80 | 2.50 | 1.32 | 0.95 | 0.97 | 1.08 | 2.36 | 1.85 | 3.94 |

The results showed that AMP-33 exhibited a strong inhibitory effect on pathogenic microorganisms and also inhibited the expression of inflammatory factors induced by LPS in RAW264.7 cells.

### Example 8

This study aimed to test the bioactivity of AMP-33 and to determine whether its anti-inflammatory effect on RAW264.7 cells is diminished after high-temperature treatment.

### Experimental Steps:

(1) The peptides were dissolved in water to a concentration of 2 mg/mL and incubated at 40° C. for 40 days for high-temperature treatment.

(2) Mouse macrophage RAW264.7 cells were cultured in DMEM supplemented with 10% fetal bovine serum (FBS) and 1% penicillin and streptomycin (P/S) at 37° C. in a 5% CO₂ incubator.

(3) 5×10⁴ mouse-derived RAW264.7 macrophages were cultured in 12-well cell culture plates for 24 hours. Then, 20 μL of PBS (negative control), Dexamethasone (positive control), and the test peptide were added to the wells, and the cells were cultured for an additional 4 hours. After that, 10 ng/ml of lipopolysaccharide (LPS) was added to the RAW264.7 cells, and the mixture was incubated for 24 hours.

(4) The supernatant was collected, and the NO level was measured using a Biotin NO Assay Kit. The OD value was measured, and the NO content was calculated according to the standard curve.

The results are shown in FIG. 8, indicating that the peptide is not inactivated after high-temperature treatment and exhibits good thermal stability.

It will be apparent to those skilled in the art that changes and modifications may be made. Therefore, the intent of the appended claims is to cover all such changes and modifications.

What is claimed is:

1. A peptide or a derivative thereof, comprising one of the following sequences:
an amino acid sequence of SEQ ID NO: 1, or a variant thereof; and
an amino acid sequence of SEQ ID NO: 2, or a variant thereof.

2. The peptide or a derivative thereof according to claim 1, wherein the variant of SEQ ID NO: 1 has ≥88% sequence identity with SEQ ID NO: 1, and the variant of SEQ ID NO: 2 has ≥88% sequence identity with SEQ ID NO: 2.

3. The peptide or a derivative thereof according to claim 1, wherein the variant of SEQ ID NO: 1 has an amino acid sequence of SEQ ID NO: 3 or SEQ ID NO: 4.

4. The peptide or a derivative thereof according to claim 1, wherein variation(s) in SEQ ID NO: 1 consists only of one or more conservative amino acid substitutions among amino acids within each of the following groups: (a) glycine, alanine, valine, leucine, and isoleucine; (b) phenylalanine, tyrosine, and tryptophan; (c) serine and threonine; (d) aspartate and glutamate; (e) glutamine and asparagine; and (f) lysine, arginine and histidine.

5. The peptide or a derivative thereof according to claim 1, wherein each of the amino acid sequences of SEQ ID NO: 1 and SEQ ID NO: 2 comprises an N-terminal; a hydrogen atom of the N-terminal is substituted by CH₃CH(OH)CO— or R₁—CO—, and R₁ is selected from a group consisting of H, hydroxyl, amino, an alkyl group, and an alkenyl group.

6. The peptide or a derivative thereof according to claim 1, wherein each of the amino acid sequences of SEQ ID NO: 1 and SEQ ID NO: 2 comprises a C-terminal; a hydrogen atom of the C-terminal is substituted by —NR₂R₃ or —OR₂, and R₂ and R₃ are independently selected from a group consisting of H, hydroxyl, amino, an alkyl group, and an alkenyl group.

7. The peptide or a derivative thereof according to claim 5, wherein the alkyl group is selected from a group consisting of methyl, ethyl, isopropyl, isobutyl, tert-butyl, pentyl, hexyl, heptyl, octyl, decyl, 2-ethylhexyl, 2-methylbutyl, and 5-methylhexyl.

8. The peptide or a derivative thereof according to claim 6, wherein the alkyl group is selected from a group consisting of methyl, ethyl, isopropyl, isobutyl, tert-butyl, pentyl, hexyl, heptyl, octyl, decyl, 2-ethylhexyl, 2-methylbutyl, and 5-methylhexyl.

9. The peptide or a derivative thereof according to claim 5, wherein the alkenyl group is selected from a group consisting of vinyl, linoleyl, and oleyl.

10. The peptide or a derivative thereof according to claim 6, wherein the alkenyl group is selected from a group consisting of vinyl, linoleyl, and oleyl.

11. A pharmaceutical composition, comprising:
a medication container; and
a therapeutically effective amount of the peptide or a derivative thereof of claim 1.

12. The pharmaceutical composition of claim 11, wherein the peptide or a derivative thereof is in solid powder form in an amount of 200-2,500 mg per dose.

13. The pharmaceutical composition of claim 11, further comprising an aqueous solution contained in the medication container; wherein the peptide or a derivative thereof is dissolved in the aqueous solution at a concentration of 3-30 mg/mL.

14. A method for treating a medical condition in a patient, comprising administering to the patient a therapeutically effective amount of the peptide or a derivative thereof of claim 1.

15. The method of claim 14, wherein an amount of the peptide or a derivative thereof is administered as a single dose is 200-2,500 mg per dose or 2-60 mg/kg of body weight.

16. The method of claim 14, wherein the medical condition is acne and the peptide or a derivative thereof is administered topically to a treatment area on the patient's skin.

17. A method for preparing a cosmetically or pharmaceutically acceptable salt comprising applying the peptide or a derivative thereof of claim 1.

18. The method of claim 17, wherein the salt is formed by the peptide or a derivative thereof of claim 1 with an organic base, and the organic base is ethylamine, diethylamine, arginine, lysine, histidine or piperazine.

19. The method of claim 17, wherein the salt is formed by the peptide or a derivative thereof of claim 1 with an inorganic acid or an organic acid; the organic acid is acetic acid, citric acid, malonic acid, maleic acid, tartaric acid, fumaric acid, benzoic acid, succinic acid, oxalic acid, or gluconic acid; and the inorganic acid is hydrochloric acid, sulphuric acid, boric acid or carbonic acid.


SEQUENCE LISTING (6 sequences)

SEQ ID NO: 1 - protein, 15 aa, synthetic construct
GIFKKITGKLFKWIK

SEQ ID NO: 2 - protein, 14 aa, synthetic construct
LSKWLKKLGKLLAG

SEQ ID NO: 3 - protein, 15 aa, synthetic construct
GIFKKITGKCFKWIK

SEQ ID NO: 4 - protein, 15 aa, synthetic construct
GIFKKIAGKLFKWIK

SEQ ID NO: 5 - protein, 14 aa, synthetic construct
FPLTWLKWWKWKKK

SEQ ID NO: 6 - protein, 17 aa, synthetic construct
FKRLKKLFKKKKIWNWK